(19) United States
(12) Patent Application Publication
(10) Pub. No.: US 2013/0337092 A1
(43) Pub. Date: Dec. 19, 2013

(54) PHYTONUTRIENT COMPOSITIONS AND METHODS OF USE

(71) Applicant: Metaproteomics, LLC, Aliso Viejo, CA (US)

(72) Inventors: Veera KONDA, Gig Harbor, WA (US); Andrea Lyra HELLER, Gig Harbor, WA (US); Anu DESAI, Gig Harbor, WA (US); Brian CARROLL, Gig Harbor, WA (US); Matthew L. TRIPP, Gig Harbor, WA (US); Jeffrey S. BLAND, Fox Island, WA (US)

(73) Assignee: METAPROTEOMICS, LLC, Aliso Viejo, CA (US)

(21) Appl. No.: 13/897,080

(22) Filed: May 17, 2013

Related U.S. Application Data
(63) Continuation-in-part of application No. 13/715,794, filed on Dec. 14, 2012.
(60) Provisional application No. 61/648,814, filed on May 18, 2012.
(60) Provisional application No. 61/576,158, filed on Dec. 15, 2011.

(51) Int. Cl. A61K 36/87; A61K 36/48; A61K 36/28; A61K 36/53; A61K 36/736; A61K 36/54; A61K 31/366; A61K 31/216; A61K 36/31; A61K 36/42; A61K 31/353; A61K 36/45; A61K 31/7048

(52) U.S. Cl. CPC A61K 36/87; A61K 36/45; A61K 36/48; A61K 36/28; A61K 36/53; A61K 36/736; A61K 31/7048; A61K 31/366; A61K 31/216; A61K 36/31; A61K 36/42; A61K 31/353; A61K 36/54
USPC 424/732

(57) ABSTRACT

Phytonutrient based compositions and methods of using the same for preventing, reducing, or treating genetic damage induced by environmental toxins are disclosed. Additionally, said compositions and methods are used to reduce oxidized low density lipoproteins (OxLDL), myeloperoxidase (MPO), and plasminogen activator inhibitor-1 (PAI-1).

### CROSS-REFERENCE TO RELATED APPLICATIONS

This patent application is a continuation-in-part of U.S. application Ser. No. 13/715,794, filed Dec. 14, 2012, which claims priority to U.S. provisional application No. 61/576,158, filed Dec. 15, 2011. This application also claims the priority benefit of U.S. provisional application No. 61/648,814, filed May 18, 2012. Each of the above prior applications is hereby expressly incorporated herein by reference in its entirety.

### BACKGROUND OF THE INVENTION

1. Field of the Invention

The present invention relates to phytonutrient compositions and methods of using the same for preventing, reducing, or treating genetic damage induced by environmental agents. Additionally, said compositions and methods may be used to reduce oxidized low density lipoproteins (OxLDL), myeloperoxidase (MPO), and plasminogen activator inhibitor-1 (PAI-1).

2. Description of the Related Art

A consequence of our modern industrial society has been the proliferation of chemicals and agents released into the environment having the capacity to induce genetic damage. The list of such chemicals and agents is extensive and grows at an ever increasing pace. Some examples of these agents include heavy metals, PCBs, pesticides, volatile organic compounds, phthalates, chlorine, dioxins, chloroform, and asbestos. Modern building methods stressing air tightness and energy efficiency have now made molds a problematic concern.

The list of offending compounds grows daily, in large part, due to recent advances and enhanced sensitivity in detecting DNA damage caused by such environmental agents. This is especially true where assays are now conducted at the individual cell level.

DNA damage can occur naturally and is usually repaired via natural repair processes in the body. It is when the rate of damage exceeds the body's ability to keep up with repairs that health problems ensue. Some health problems associated with environmental toxin exposure include liver and kidney damage (chloroform), mesotheilioma (asbestos), reproductive damage (dioxins), and nerve damage (pesticides). Additionally, some disorders including cerebral palsy, ADHD, epilepsy, and Tourette syndrome have been ascribed to fetal exposure to various chemicals.

As the list of offending agents increases, one questions whether any lifestyle changes in today's society can alleviate the risks entirely. Today, this seems an unlikely scenario as many of the offending agents have become integral to our way of life.

What is therefore needed are means to prevent DNA damage from occurring initially or, alternatively, reduce or correct the damage subsequent to damage initiation to prevent the accumulation of DNA damage from reaching a level of health concerns.

Atherosclerosis, otherwise known as hardening and narrowing of the arteries is a progressive process which silently and slowly blocks arteries, thereby placing blood flow at risk. It is also the usual cause of heart attacks, strokes, and peripheral vascular disease, also known as cardiovascular disease.” Cardiovascular disease is the No. 1 killer in America, with more than 800,000 deaths in 2005.

Arterial wall macrophages can accumulate low density lipoproteins (LDL) where such accumulation serves to trigger atherosclerosis. High density lipoproteins (HDL) conversely can promote cholesterol efflux from macrophage by the membrane associated ATP-binding cassette transporter A1 pathway. The loss of the protective effect of HDL is poorly understood but has been attributed to oxidative damage of the HDL by myeloperoxidase (MPO) (Shao, B., et al. Chem Res Toxicol (15):447-454; 2010). Further, MPO has been argued as being a pathophysiologically distinct, independent predictor of risk in acute coronary syndromes (Armstrong, E., et al., Circulation (113), 289-292; 2006).

Increased LDL oxidation is associated with coronary artery disease. Oxidized LDL (OxLDL) induces atherosclerosis through stimulation of monocyte infiltration and smooth muscle cell migration and proliferation. OxLDL contributes to atherothrombosis by inducing endothelial cell apoptosis resulting in plaque erosion, and by impairing the anticoagulant balance in endothelium, stimulating tissue factor production by smooth muscle cells, and inducing apoptosis in macrophages (Mertens, a. and Holvoet, P. FASEB Journal (15):2073-2084; 2001).

Additionally, plasminogen activator inhibitor 1 (PAI-1) plays a role in atherosclerosis by modulating clot formation and resorption. As such, reducing PAI-1, in conjunction with MPO and OxLDL, should interact to produce beneficial effects for the prevention and control of cardiovascular disease.

### SUMMARY OF THE INVENTION

The present invention relates to phytonutrient compositions and methods of using the same for preventing, reducing, or treating genetic damage induced by environmental agents. Additionally, said compositions and methods are used to reduce oxidized low density lipoproteins (OxLDL), myeloperoxidase (MPO), and plasminogen activator inhibitor-1 (PAI-1).

A first embodiment of the invention describes phytonutrient compositions for preventing, reducing, or treating genetic damage induced by environmental agents, where the compositions comprise therapeutically effective amounts of two or more compounds selected from the group consisting of Acacia nilotica extract, artichoke extract, blueberry extract, catechins, chlorogenic acid, cinnamon, ellagic acid, grape seed extract, hesperidin, Momodica extract, prune extract, rosemary extract, and watercress extract and the composition is in a dosage form suitable for oral administration.

A second embodiment of the invention describes methods for preventing, reducing, or treating genetic damage induced by environmental agents, where the methods comprise administering a composition comprising therapeutically effective amounts of two or more compounds selected from the group consisting of Acacia nilotica extract, artichoke extract, blueberry extract, catechins, chlorogenic acid, cinnamon, ellagic acid, grape seed extract, hesperidin, Momodica extract, prune extraci, rosemary extract, and watercress extract and the composition is in a dosage form suitable for oral administration.

A third embodiment of the invention describes phytonutrient compositions for reducing OxLDL, MPO, and PAI-1, where the compositions comprise therapeutically effective amounts of two or more compounds selected from the group consisting of Acacia nilotica extract, artichoke extract, blueberry extract, catechins, chlorogenic acid, cinnamon, ellagic acid, grape seed extract, hesperidin, Momodica extract, prune extract, rosemary extract, and watercress extract and the composition is in a dosage form suitable for oral administration.

A fourth embodiment of the invention describes the use of a composition for simultaneously reducing serum OxLDL, MPO, and PAI-1 levels in a subject in need thereof, wherein said use comprises administering a composition comprising therapeutically effective amounts of two or more compounds or extracts selected from the group consisting of Acacia nilotica extract, artichoke extract, blueberry extract, catechins, chlorogenic acid, cinnamon, ellagic acid, grape seed extract, hesperidin, Momodica extract, prune extract, rosemary extract, and watercress extract and said composition is in a dosage form suitable for oral administration wherein said dosage form provides:
a. from 0.5 mg to 10 mg of Acacia nilotica extract;
b. from 0.5 mg to 10 mg of artichoke extract;
c. from 1 mg to 25 mg of blueberry extract;
d. from 2.5 mg to 25 mg of catechins;
e. from 25 mg to 75 mg of chlorogenic acid;
f. from 0.5 mg to 10 mg of cinnamon;
g. from 10 mg to 100 mg of ellagic acid;
h. from 2.5 mg to 50 mg of grape seed extract;
i. from 15 mg to 120 mg of hesperidin;
j. from 0.5 mg to 10 mg of Momodica extract;
k. from 0.5 mg to 10 mg of prune extract;
l. from 0.5 mg to 10 mg of rosemary extract; and
m. from 0.5 mg to 10 mg of watercress extract.

### BRIEF DESCRIPTION OF THE DRAWINGS

FIG. 1: FIG. 1A: A graphic representation of the Tail Moment for cells treated with OG5459 as measured in the Trevigen, Inc., CometAssay®. FIG. 1B: Table of date used for generation of Panel A graphic representation.

FIG. 2: FIG. 2A: A graphic representation of the % DNA in the tail for cells treated with OG5459 as measured in the Trevigen, Inc., CometAssay®. FIG. 2B: Table of date used for generation of Panel A graphic representation.

FIG. 3: FIG. 3A: A graphic representation of the Tail Moment for cells treated with OG5507 as measured in the Trevigen, Inc., CometAssay®. FIG. 3B: Table of date used for generation of Panel A graphic representation.

FIG. 4: FIG. 4A: A graphic representation of the % DNA in the tail for cells treated with OG5507 as measured in the Trevigen, Inc., CometAssay®. FIG. 4B: Table of date used for generation of Panel A graphic representation.

FIG. 5: FIGS. 5A and 5B: Representative images for cells treated with OG5459 as measured in the Trevigen, Inc., CometAssay®.

FIG. 6: FIGS. 6A and 6B: Representative images for cells treated with OG5507 as measured in the Trevigen, Inc., CometAssay®.

FIG. 7: Representative effects of four week supplementation with phytonutrient composition of the invention on reduced serum AxLDL, PAI-1 and MPO levels in humans. (*p<0.05 compared with V2 vs. V3

### DETAILED DESCRIPTION OF THE INVENTION

The present invention relates to phytonutrient compositions and methods of using the same for preventing, reducing, or treating genetic damage induced by environmental agents. Additionally, said compositions and methods may be used to reduce oxidized low density lipoproteins (OxLDL), myeloperoxidase (MPO), and plasminogen activator inhibitor-1 (PAI-1).

The patents, published applications, and scientific literature referred to herein establish the knowledge of those with skill in the art and are hereby incorporated by reference in their entirety to the same extent as if each was specifically and individually indicated to be incorporated by reference. Any conflict between any reference cited herein and the specific teachings of this specification shall be resolved in favor of the latter. Likewise, any conflict between an art-understood definition of a word or phrase and a definition of the word or phrase as specifically taught in this specification shall be resolved in favor of the latter.

Technical and scientific terms used herein have the meaning commonly understood by one of skill in the art to which the present invention pertains, unless otherwise defined. Reference is made herein to various methodologies and materials known to those of skill in the art. Standard reference works setting forth the general principles of recombinant DNA technology include Sambrook et al., Molecular Cloning: A Laboratory Manual, 2nd Ed., Cold Spring Harbor Laboratory Press, New York (1989); Kaufman et al., Eds., Handbook of Molecular and Cellular Methods in Biology in Medicine, CRC Press, Boca Raton (1995); McPherson, Ed., Directed Mutagenesis: A Practical Approach, IRL Press, Oxford (1991). Standard reference works setting forth the general principles of pharmacology include Goodman and Gilman's The Pharmacological Basis of Therapeutics, 11th Ed., McGraw Hill Companies Inc., New York (2006).

In the specification and the appended claims, the singular forms include plural referents unless the context clearly dictates otherwise. As used in this specification, the singular forms “a,” “an” and “the” specifically also encompass the plural forms of the terms to which they refer, unless the content clearly dictates otherwise. Additionally, as used herein, unless specifically indicated otherwise, the word “or” is used in the “inclusive” sense of “and/or” and not the “exclusive” sense of “either/or.” The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

As used herein, the recitation of a numerical range for a variable is intended to convey that the invention may be practiced with the variable equal to any of the values within that range. Thus, for a variable that is inherently discrete, the variable can be equal to any integer value of the numerical range, including the end-points of the range. Similarly, for a variable that is inherently continuous, the variable can be equal to any real value of the numerical range, including the end-points of the range. As an example, a variable that is described as having values between 0 and 2, can be 0, 1 or 2 for variables that are inherently discrete, and can be 0.0, 0.1, 0.01, 0.001, or any other real value for variables that are inherently continuous.

Reference is made hereinafter in detail to specific embodiments of the invention. While the invention will be described in conjunction with these specific embodiments, it will be understood that it is not intended to limit the invention to such specific embodiments. On the contrary, it is intended to cover alternatives, modifications, and equivalents as may be included within the spirit and scope of the invention as defined by the appended claims. In the following description, numerous specific details are set forth in order to provide a thorough understanding of the present invention. The present invention may be practiced without some or all of these specific details. In other instances, well known process operations have not been described in detail, in order not to unnecessarily obscure the present invention.

A first embodiment of the invention describes phytonutrient compositions for preventing, reducing, or treating genetic damage induced by environmental agents, where the compositions comprise therapeutically effective amounts of two or more compounds selected from the group consisting of Acacia nilotica extract, artichoke extract, blueberry extract, catechins, chlorogenic acid, cinnamon, ellagic acid, grape seed extract, hesperidin, Momodica extract, prune extract, rosemary extract, and watercress extract and the composition is in a dosage form suitable for oral administration.

In an aspect of this embodiment, the composition provides per dosage form:
a. from 0.5 mg to 10 mg of Acacia nilotica extract;
b. from 0.5 mg to 10 mg of artichoke extract;
c. from 1 mg to 25 mg of blueberry extract;
d. from 2.5 mg to 25 mg of catechins;
e. from 25 mg to 75 mg of chlorogenic acid;
f. from 0.5 mg to 10 mg of cinnamon;
g. from 10 mg to 100 mg of ellagic acid;
h. from 2.5 mg to 50 mg of grape seed extract;
i. from 15 mg to 120 mg of hesperidin;
j. from 0.5 mg to 10 mg of Momodica extract;
k. from 0.5 mg to 10 mg of prune extract;
l. from 0.5 mg to 10 mg of rosemary extract; and
m. from 0.5 mg to 10 mg of watercress extract.

As used herein, a “phytonutrient” refers to nutrients derived from plant materials shown to be necessary for sustaining human life. Encompassed within phytonutrients are “phytochemicals”, that is, plant derived compounds not yet classified as substances needed for sustaining life yet known to have properties useful for aiding in the prevention of disease.

As used herein, “genetic damage” refers to damage to the cell's DNA (either physical or in the sense of its information capacity) when it mutates (or deviates) from its original form. There are many known substances which can damage DNA, such as, for example, oxidizing agents, alkylating agents and electromagnetic radiation. Damage by these agents includes cross linking between the pyrimidine bases, base modifications and double strand breaks.

As used herein, the term “extract” refers to the solid material resulting from (1) exposing a plant material to a solvent, (2) separating the solvent from the plant material, and (3) eliminating the solvent.

As used herein, the term “solvent” refers to a liquid of aqueous or organic nature possessing the necessary characteristics to extract solid material from plant material. Examples of solvents would include water, steam, superheated water, methanol, ethanol, hexane, chloroform, liquid CO₂, liquid N₂ or any combinations of such materials.

The term “acacia”, as used herein, refers to any member of leguminous trees and shrubs of the genus Acacia. Preferably, the botanical compound derived from acacia is derived from Acacia catechu or Acacia nilotica. In those aspects where the acacia derived compound or extract is derived from Acacia catechu or Acacia nilotica, the Acacia catechu or Acacia nilotica compound is selected from the group consisting of gum resin, bark powder, heartwood powder, and an Acacia catechu or Acacia nilotica extract. In those aspects where the acacia derived compound is an Acacia catechu or Acacia nilotica extract, the extract is selected from acidic, alkaline, polar solvent, nonpolar solvent, and gastric fluid extracts.

As used herein, by “treating” is meant reducing, preventing, and/or reversing the symptoms in the individual to which a compound of the invention has been administered, as compared to the symptoms of an individual not being treated according to the invention. A practitioner will appreciate that the compounds, compositions, and methods described herein are to be used in concomitance with continuous clinical evaluations by a skilled practitioner (physician or veterinarian) to determine subsequent therapy. Hence, following treatment the practitioners will evaluate any improvement in the treatment of the pulmonary inflammation according to standard methodologies. Such evaluation will aid and inform in evaluating whether to increase, reduce or continue a particular treatment dose, mode of administration, etc.

It will be understood that the subject to which a compound of the invention is administered need not suffer from a specific traumatic state. Indeed, the compounds of the invention may be administered prophylactically, prior to any development of symptoms. The term “therapeutic,” “therapeutically,” and permutations of these terms are used to encompass therapeutic, palliative as well as prophylactic uses. Hence, as used herein, by “treating or alleviating the symptoms” is meant reducing, preventing, and/or reversing the symptoms of the individual to which a compound of the invention has been administered, as compared to the symptoms of an individual receiving no such administration.

The term “therapeutically effective amount” is used to denote treatments at dosages effective to achieve the therapeutic result sought. Furthermore, one of skill will appreciate that the therapeutically effective amount of the compound of the invention may be lowered or increased by fine tuning and/or by administering more than one compound of the invention, or by administering a compound of the invention with another compound. See, for example, Meiner, C. L., “Clinical Trials: Design, Conduct, and Analysis,” Monographs in Epidemiology and Biostatistics, Vol. 8 Oxford University Press, USA (1986). The invention therefore provides a method to tailor the administration/treatment to the particular exigencies specific to a given mammal. As illustrated in the following examples, therapeutically effective amounts may be easily determined for example empirically by starting at relatively low amounts and by step-wise increments with concurrent evaluation of beneficial effect.

It will be appreciated by those of skill in the art that the number of administrations of the compounds according to the invention will vary from patient to patient based on the particular medical status of that patient at any given time including other clinical factors such as age, weight and condition of the mammal and the route of administration chosen.

As used herein, “compounds” may be identified either by their chemical structure, chemical name, or common name. When the chemical structure and chemical or common name conflict, the chemical structure is determinative of the identity of the compound. The compounds described herein may contain one or more chiral centers and/or double bonds and therefore, may exist as stereoisomers, such as double-bond isomers (i.e., geometric isomers), enantiomers or diastereomers. Accordingly, the chemical structures depicted herein encompass all possible enantiomers and stereoisomers of the illustrated or identified compounds including the stereoisomerically pure form (e.g., geometrically pure, enantiomerically pure or diastereomerically pure) and enantiomeric and stereoisomeric mixtures. Enantiomeric and stereoisomeric mixtures can be resolved into their component enantiomers or stereoisomers using separation techniques or chiral synthesis techniques well known to the skilled artisan. The compounds may also exist in several tautomeric forms including the enol form, the keto form and mixtures thereof. Accordingly, the chemical structures depicted herein encompass all possible tautomeric forms of the illustrated or identified compounds. The compounds described also encompass isotopically labeled compounds where one or more atoms have an atomic mass different from the atomic mass conventionally found in nature. Examples of isotopes that may be incorporated into the compounds of the invention include, but are not limited to, ²H, ³H, ¹³C, ¹⁴C, ¹⁵N, ¹⁸O, ¹⁷O, etc. Compounds may exist in unsolvated forms as well as solvated forms, including hydrated forms and as N-oxides. In general, compounds may be hydrated, solvated or N-oxides. Certain compounds may exist in multiple crystalline or amorphous forms. Also contemplated within the scope of the invention are congeners, analogs, hydrolysis products, metabolites and precursor or prodrugs of the compound. In general, unless otherwise indicated, all physical forms are equivalent for the uses contemplated herein and are intended to be within the scope of the present invention.

Compounds according to the invention may be present as salts. In particular, pharmaceutically acceptable salts of the compounds are contemplated. A “pharmaceutically acceptable salt” of the invention is a combination of a compound of the invention and either an acid or a base that forms a salt (such as, for example, the magnesium salt, denoted herein as “Mg” or “Mag”) with the compound and is tolerated by a subject under therapeutic conditions. In general, a pharmaceutically acceptable salt of a compound of the invention will have a therapeutic index (the ratio of the lowest toxic dose to the lowest therapeutically effective dose) of 1 or greater. The person skilled in the art will recognize that the lowest therapeutically effective dose will vary from subject to subject and from indication to indication, and will thus adjust accordingly.

The compounds according to the invention are optionally formulated in a pharmaceutically acceptable vehicle with any of the well known pharmaceutically acceptable carriers, including diluents and excipients [see Remington's Pharmaceutical Sciences, 18th Ed., Gennaro, Mack Publishing Co., Easton, Pa. 1990 and Remington: The Science and Practice of Pharmacy, Lippincott, Williams & Wilkins, 1995]. While the type of pharmaceutically acceptable carrier/vehicle employed in generating the compositions of the invention will vary depending upon the mode of administration of the composition to a mammal, generally pharmaceutically acceptable carriers are physiologically inert and non-toxic. Formulations of compositions according to the invention may contain more than one type of compound of the invention), as well as any other pharmacologically active ingredient useful for the treatment of the symptom/condition being treated.

The compounds of the present invention may be provided in a pharmaceutically acceptable vehicle using formulation methods known to those of ordinary skill in the art. The compositions of the invention can be administered by standard routes, though preferably administration is by inhalation routes. The compositions of the invention include those suitable for oral, inhalation, rectal, ophthalmic (including intravitreal or intracameral), nasal, topical (including buccal and sublingual), vaginal, or parenteral (including subcutaneous, intramuscular, intravenous, intradermal, and intratracheal). In addition, polymers may be added according to standard methodologies in the art for sustained release of a given compound.

Formulations suitable for administration by inhalation include formulations that can be dispensed by inhalation devices known to those in the art. Such formulations may include carriers such as powders and aerosols. The present invention encompasses liquid and powdered compositions suitable for nebulization and intrabronchial use, or aerosol compositions administered via an aerosol unit dispensing metered doses (“MDI”). The active ingredient may be formulated in an aqueous pharmaceutically acceptable inhalant vehicle, such as, for example, isotonic saline or bacteriostatic water and other types of vehicles that are well known in the art. The solutions are administered by means of a pump or squeeze-actuated nebulized spray dispenser, or by any other conventional means for causing or enabling the requisite dosage amount of the liquid composition to be inhaled into the patient's lungs. Powder compositions containing the anti-inflammatory compounds of the present invention include, by way of illustration, pharmaceutically acceptable powdered preparations of the active ingredient thoroughly intermixed with lactose or other inert powders acceptable for intrabronchial administration. The powder compositions can be administered via a dispenser, including, but not limited to, an aerosol dispenser or encased in a breakable capsule which may be inserted by the patient into a device that punctures the capsule and blows the powder out in a steady stream. Aerosol formulations for use in the subject method typically include propellants, surfactants, and co-solvents and may be filled into conventional aerosol containers that are closed by a suitable metering valve.

Formulations of compositions of the present invention suitable for nasal administration, wherein the carrier is a solid, include a coarse powder having a particle size, for example, in the range of 20 to 500 microns which is administered in the manner in which snuff is administered, i.e., by rapid inhalation through the nasal passage from a container of the powder held close up to the nose. Suitable formulations, wherein the carrier is a liquid, for administration, for example via a nasal spray, aerosol, or as nasal drops, include aqueous or oily solutions of the compound of the invention.

For oral administration, the compositions of the invention may be presented as discrete units such as capsules, caplets, gelcaps, cachets, pills, or tablets each containing a predetermined amount of the active ingredient as a powder or granules; as a solution or a suspension in an aqueous liquid or a non-aqueous liquid; or as an oil-in-water liquid emulsion or a water-in-oil emulsion and as a bolus, etc. Alternately, administration of a composition of all of the aspects of the present invention may be effected by liquid solutions, suspensions or elixirs, powders, lozenges, micronized particles and osmotic delivery systems.

Formulations of compositions according to the aspects of the present invention suitable for parenteral administration include aqueous and non-aqueous sterile injection solutions which may contain antioxidants, stabilizers, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze-dried (lyophilized) conditions requiring only the addition of the sterile liquid carrier, for example, water for injections, immediately prior to use. Extemporaneous injection solutions and suspensions may be prepared from sterile powders, granules and tablets of the kind previously described.

A tablet may be made by compression or molding, optionally with one or more accessory ingredients. Compressed tablets may be prepared by compressing, in a suitable machine, the active ingredient in a free-flowing form such as a powder or granules, optionally mixed with a binder, lubricant, inert diluent, preservative, surface active or dispersing agent. Molded tablets may be made by molding, in a suitable machine, a mixture of the powdered compound moistened with an inert liquid diluent. The tablets may be optionally coated or scored and may be formulated to provide a slow or controlled release of the active ingredient therein.

Formulations of compositions of the present invention for rectal administration may be prepared as a suppository with a suitable base comprising, such as, for example, cocoa butter.

Formulations of compositions of the present invention suitable for topical administration in the mouth include lozenges comprising the ingredients in a flavored basis, usually sucrose and acacia or tragacanth; pastilles comprising the active ingredient in an inert basis such as gelatin and glycerin, or sucrose and acacia; and mouthwashes comprising the ingredient to be administered in a suitable liquid carrier. Formulations of compositions of the present invention suitable for topical administration to the skin may be presented as ointments, creams, gels, lotions and pastes comprising the ingredient to be administered in a pharmaceutical acceptable carrier. A topical delivery system contemplated is a transdermal patch containing the ingredient to be administered.

Formulations of compositions according to the aspects of the present invention suitable for vaginal administration may be presented as pessaries, suppositories, tampons, creams, gels, pastes, foams or spray formulations containing in addition to the compound of the invention such pharmaceutically acceptable carriers as are known in the art to be appropriate.

The methods and compositions of the present invention are intended for use with any mammal that may experience the benefits of the methods of the invention. Foremost among such mammals are humans, although the invention is not intended to be so limited, and is applicable to veterinary uses. Thus, in accordance with the invention, “mammals” or “mammal in need” include humans as well as non-human mammals, particularly domesticated animals including, without limitation, cats, dogs, and horses.

A second embodiment of the invention describes methods for preventing, reducing, or treating genetic damage induced by environmental agents, where the methods comprise administering a composition comprising therapeutically effective amounts of two or more compounds selected from the group consisting of Acacia nilotica extract, artichoke extract, blueberry extract, catechins, chlorogenic acid, cinnamon, ellagic acid, grape seed extract, hesperidin, Momodica extract, prune extract, rosemary extract, and watercress extract and the composition is in a dosage form suitable for oral administration.

In an aspect of this embodiment, the composition of the method provides per dosage form:
a. from 0.5 mg to 10 mg of Acacia nilotica extract;
b. from 0.5 mg to 10 mg of artichoke extract;
c. from 1 mg to 25 mg of blueberry extract;
d. from 2.5 mg to 25 mg of catechins;
e. from 25 mg to 75 mg of chlorogenic acid;
f. from 0.5 mg to 10 mg of cinnamon;
g. from 10 mg to 100 mg of ellagic acid;
h. from 2.5 mg to 50 mg of grape seed extract;
i. from 15 mg to 120 mg of hesperidin;
j. from 0.5 mg to 10 mg of Momodica extract;
k. from 0.5 mg to 10 mg of prune extract;
l. from 0.5 mg to 10 mg of rosemary extract; and
m. from 0.5 mg to 10 mg of watercress extract.

A third embodiment of the invention describes phytonutrient compositions for reducing OxLDL, MPO, and PAI-1, where the compositions comprise therapeutically effective amounts of two or more compounds selected from the group consisting of Acacia nilotica extract, artichoke extract, blueberry extract, catechins, chlorogenic acid, cinnamon, ellagic acid, grape seed extract, hesperidin, Momodica extract, prune extract, rosemary extract, and watercress extract and the composition is in a dosage form suitable for oral administration.

In an aspect of this embodiment, the composition provides per dosage form:
a. from 0.5 mg to 10 mg of Acacia nilotica extract;
b. from 0.5 mg to 10 mg of artichoke extract;
c. from 1 mg to 25 mg of blueberry extract;
d. from 2.5 mg to 25 mg of catechins;
e. from 25 mg to 75 mg of chlorogenic acid;
f. from 0.5 mg to 10 mg of cinnamon;
g. from 10 mg to 100 mg of ellagic acid;
h. from 2.5 mg to 50 mg of grape seed extract;
i. from 15 mg to 120 mg of hesperidin;
j. from 0.5 mg to 10 mg of Momodica extract;
k. from 0.5 mg to 10 mg of prune extract;
l. from 0.5 mg to 10 mg of rosemary extract; and
m. from 0.5 mg to 10 mg of watercress extract

A fourth embodiment of the invention describes the use of a composition for simultaneously reducing serum OxLDL, MPO, and PAI-1 levels in a subject in need thereof, wherein said use comprises administering a composition comprising therapeutically effective amounts of two or more compounds or extracts selected from the group consisting of Acacia nilotica extract, artichoke extract, blueberry extract, catechins, chlorogenic acid, cinnamon, ellagic acid, grape seed extract, hesperidin, Momodica extract, prune extract, rosemary extract, and watercress extract and said composition is in a dosage form suitable for oral administration wherein said dosage form provides:
a. from 0.5 mg to 10 mg of Acacia nilotica extract;
b. from 0.5 mg to 10 mg of artichoke extract;
c. from 1 mg to 25 mg of blueberry extract;
d. from 2.5 mg to 25 mg of catechins;
e. from 25 mg to 75 mg of chlorogenic acid;
f. from 0.5 mg to 10 mg of cinnamon;
g. from 10 mg to 100 mg of ellagic acid;
h. from 2.5 mg to 50 mg of grape seed extract;
i. from 15 mg to 120 mg of hesperidin;
j. from 0.5 mg to 10 mg of Momodica extract;
k. from 0.5 mg to 10 mg of prune extract;
l. from 0.5 mg to 10 mg of rosemary extract; and
m. from 0.5 mg to 10 mg of watercress extract.

In aspects of this embodiment, the number of compounds or extracts of the composition used preferably three or more compounds or extracts; more preferably four, five or six compounds or extracts; and most preferably seven compounds or extracts of the composition of the invention.

Any suitable materials and/or methods known to those of skill can be utilized in carrying out the present invention. However, preferred materials and methods are described. Materials, reagents and the like to which reference are made in the following description and examples are obtainable from commercial sources, unless otherwise noted.

### EXAMPLES

### Example 1

### Effects of Compositions OG5495 and OG5507 on Healthy and Hydrogen Peroxide Treated Jurkat Cells by CometAssay

Purpose: To evaluate effects of test compositions OG5495 and OG5507 on healthy and hydrogen peroxide treated Jurkat cells by CometAssay. The formulations for compositions OG5495 and OG5507 are presented in Table 1 below.

TABLE 1

| | OG5507 | OG5495 |
|---|---|---|
| Ingredient | (mg/dosage form) | (mg/dosage form) |
| Hesperidin | 61.00 | 61.00 |
| Ellagic acid | 41.25 | 41.25 |
| Prune extract | 2.50 | 2.50 |
| Watercress extract | 2.50 | 2.50 |
| Chlorogenic acid | 55.71 | 55.71 |
| Cinnamon | 2.50 | 2.50 |
| Catechins | 8.58 | 8.58 |
| Acacia nilotica extract | 2.50 | 2.50 |
| Rosemary extract | 2.50 | 2.50 |
| Momodica extract | 2.57 | 2.57 |
| Grape seed extract | 12.50 | 12.50 |
| Artichoke extract | 2.50 | 2.50 |
| Blueberry extract | 10.00 | 10.00 |
| Ascorbic acid | | 66.39 |
| Ascorbyl palmitate | | 11.16 |
| Mixed carotenoids | | 28.86 |
| Vitamin A acetate | | 6.50 |
| Biotin | | 3.00 |
| Choline bitartrate | | 41.07 |
| Methylcobalamin | | 0.13 |
| Calcium L-mefolinate | | 0.56 |
| Lycopene | | 31.50 |
| Niacin | | 6.63 |
| Niacinamide | | 20.09 |
| D-calcium pantothenate | | 52.33 |
| Phytonadione Vitamin K | | 1.32 |
| Pyridoxine hydrochloride | | 17.97 |
| Riboflavin | | 8.94 |
| Thiamin mononitrate | | 15.80 |
| Vitamin D3 | | 7.00 |
| D-alpha mixed tocopheryl | | 157.14 |
| Chromium citrate | | 5.10 |
| Copper citrate | | 3.28 |
| Manganese citrate | | 1.16 |
| Magnesium citrate | | 127.50 |
| Molybdenum aspartate | | 25.50 |
| Potassium iodide | | 7.65 |
| Selenium aspartate | | 51.00 |
| Zinc citrate dihydrate | | 24.68 |
| Inositol | | 13.53 |
| Zeaxanthin | | 22.00 |
| Lutein | | 33.00 |

The evaluation of the effects of test compositions OG5495 and OG5507 on healthy and hydrogen peroxide treated Jurkat cells was performed by Trevigen Inc. (Gaithersburg, Md.) using their CometAssay® test system. The test procedures are summarized below

Cell Treatment:

79 mg of OG5495 or 50 mg of OG5507 was suspended in DMSO. The compound solution was allowed to sit for 20 minutes at room temperature and vortex for 30 seconds every 5 minutes
a. OG5495 was clarified (5000×g, 10 minutes) and transferred to a new tube. No additional clarification was necessary for OG5495. OG5507 required no clarification.
b. Starting compound concentration was 100 mg/ml. For OG5495, this was assumed based on information provided by inventors.
Note: Remaining steps were the same for both compounds. Compound treatment was performed on separate days.

A 20 mg/ml solution was prepared by combining 10 μl 100 mg/ml stock and 40 μl of DMSO.

3×10⁵ Jurkatcells in 3 ml of RPMI (no serum) were added to eight 15 ml conical tube. One tube for each group, for a total of 8 tubes. DMSO or compound was added to each tube (as described in table below). Cells were then incubated for 1 hour at 37° C. In a 5% CO2 incubator with lose caps to allow gas exchange

3 ml of 3 × 10⁵ cells

| | | | | 10 mM | 2 mM |
|---|---|---|---|---|---|
| Sample | DMSO | 100 mg/ml | 20 mg/ml | H2O2 | H2O2 |
| DMSO Control | 3 ul | | | | |
| 20 μM H₂O₂ | 3 ul | | | 6 ul | |
| 2 μM H₂O₂ | 3 ul | | | | 3 ul |
| 100 μg/ml compound | | 3 ul | | | |
| 20 μM H2O2 + | | 3 ul | | 6 ul | |
| 100 μg/ml compound | | | | | |
| 20 μM H2O2 + | | | 3 ul | 6 ul | |
| 20 μg/ml compound | | | | | |
| 2 μM H2O2 + | | 3 ul | | | 3 ul |
| 100 μg/ml compound | | | | | |
| 2 μM H2O2 + | | | 3 ul | | 3 ul |
| 20 μg/ml compound | | | | | |

| | |
|---|---|
| 1 hr at 37° C. in 5% CO₂ | 20 minuts on ICE |

After 1 hour, samples were placed on ice. Hydrogen peroxide was added to appropriate tubes (as described in table above) and all samples were incubated on ice for 15 minutes.

1 ml of each sample was transferred to a 1.7 ml conical tube and cells were pelleted (250×g, 10 min, 4° C.). Total treatment time of 20 minutes in hydrogen peroxide.

After removing supernatant, cells were washed with 1 ml of cold 1× PBS and pelleted.

After removing the 1× PBS, cells were suspended in 1 ml freeze medium (80% RPMI, 20% FBS, 10% DMSO). A 50 μl sample aliquots were made. Samples were slowly frozen overnight at −80° C. before transferring to liquid nitrogen for storage.

Comet Assay:
1. 50 μl sample aliquots were quickly thawed and Alkaline Comet Control Cells (50 μl aliquots at 37° C.), transferred immediately to ice.
2. 500 μl of cold 1× PBS was added and spun@250×g for 5 min@4° C.
3. 525 μl of PBS was removed.
4. 50 μl of cold 1× PBS was added to pelleted cells.
5. 30 μl cells was added to 300 μl of molten LMagarose (37° C.).
6. 30 μl of LMagarose/Cell mixture per well was spread on 20 well slides. (Samples and controls are run in triplicate).
7. The slide was held@4° C. for 20 minutes.
8. Slides were transferred to Lysis Solution (pre-chilled to 4° C.) for 30 minutes at 4° C.
9. Slides were then transferred to Unwinding Solution (200 mM NaOH/1 mM EDTA) for 20 minutes at room temperature.
10. Slides were electrophoresed using Trevigen's Comet ES system using cold 200 mM NaOH/1 mM EDTA solution for 30 minutes at 21V.
11. Slides were then washed twice in water for 5 minutes at room temperature.
12. Slides were then washed in 70% Ethanol for 5 minutes at room temperature.
13. Dry slide on slide warmer (37-40° C.).
14. Add 50 μl/well of 1:30,000 SybrGold per well incubate for 30 minutes at room temperature in the dark.
15. Decant slide to remove SybrGold from slide.
16. Dip in water to quickly rinse.
17. Dry slide on slide warmer (37-40° C.).
18. Image using LOATS system

Results:

OG5495: At 100 μg/ml compound OG5495 appeared to induce DNA damage in Jurkat cells. This was demonstrated by an increase in tail moment and % DNA in the tail comparing DMSO and 100 μg/ml compound controls. Cells pretreated with 100 μAg/ml OG5495 and exposed to 20 μM H₂O₂ showed a decrease in tail moment and % DNA in the tail relative to H₂O₂ alone.

OG5507: At 100 μg/ml, compound OG5507 induced minimal damage; however, the % DNA in the tail is in the range where cells are considered healthy (less than 10% for % DNA in the tail). Unlike OG5495, OG5507 had a protective affect on Jurkat cells pretreated with 100 μg/ml of the compound before exposure to 20 μM H₂O₂. A dramatic decrease in both tail moment and % DNA in the tail was observed in cells pretreated with OG5507 compared to the untreated cells when exposed to 20 μM H₂O₂. A protective effect, although at a much lower level, was also seen in cells pretreated with 20 μg/ml of OG5507 compared to the untreated cells when exposed to 20 μM H₂O₂.

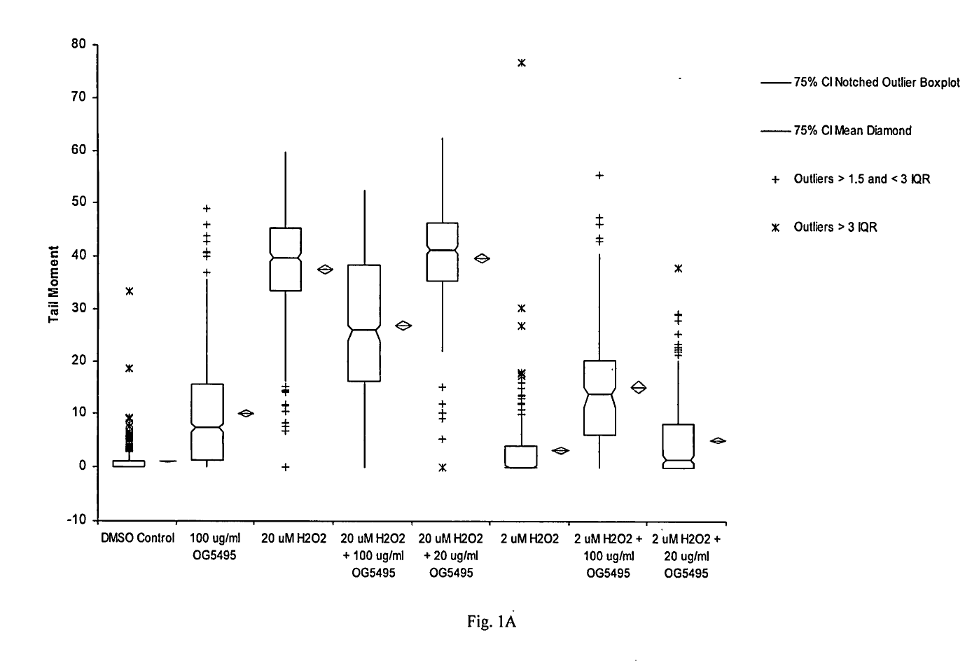

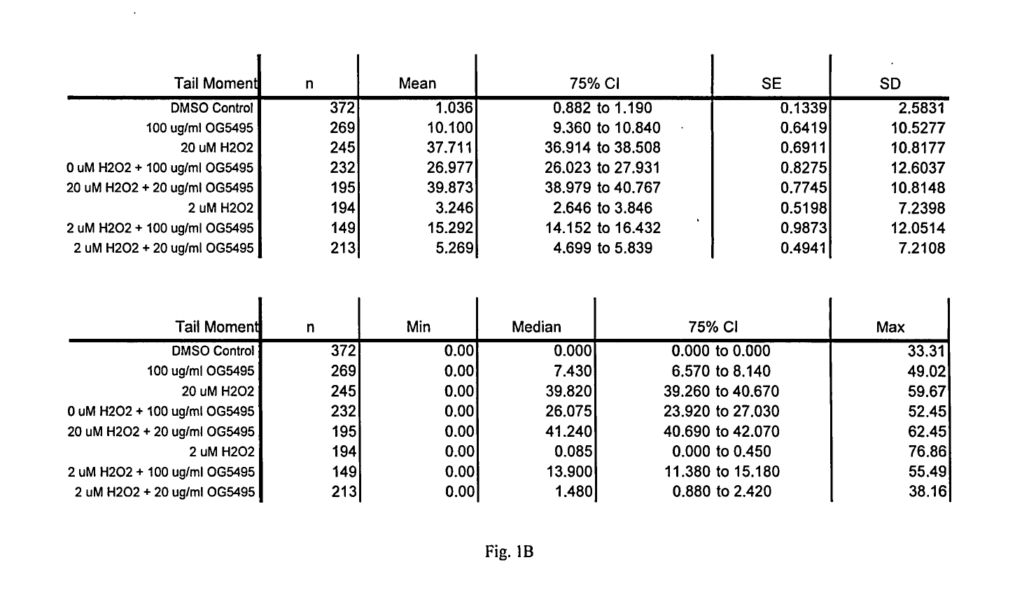

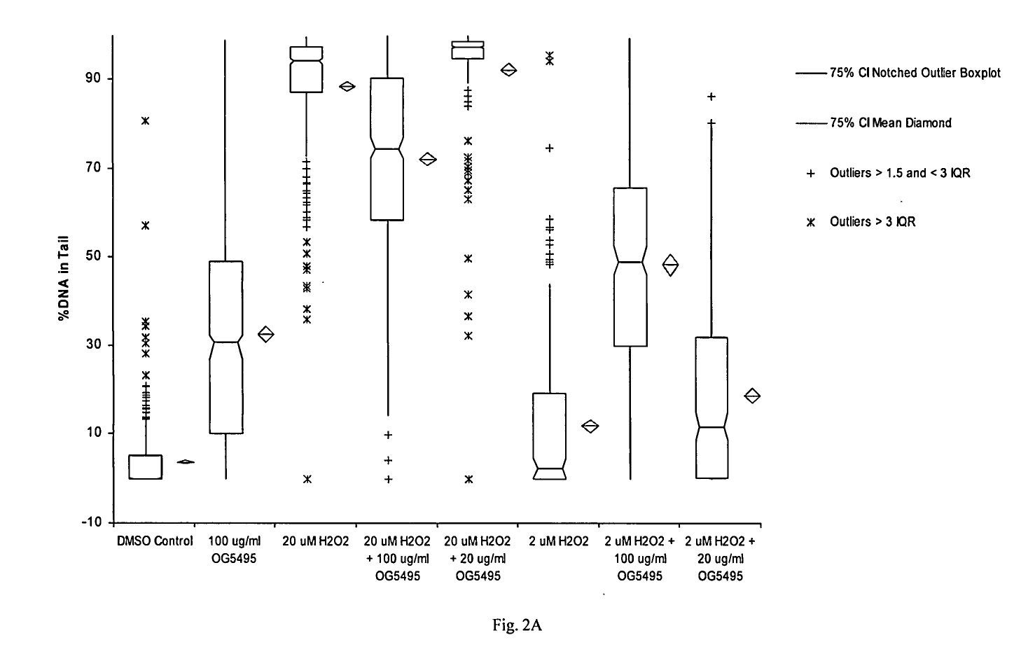

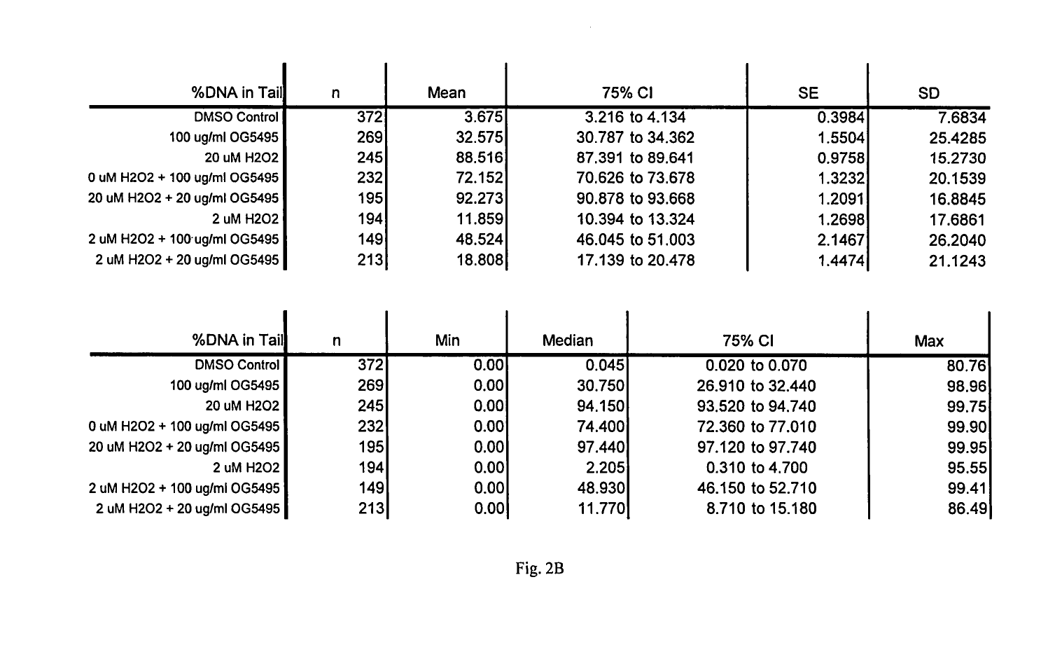

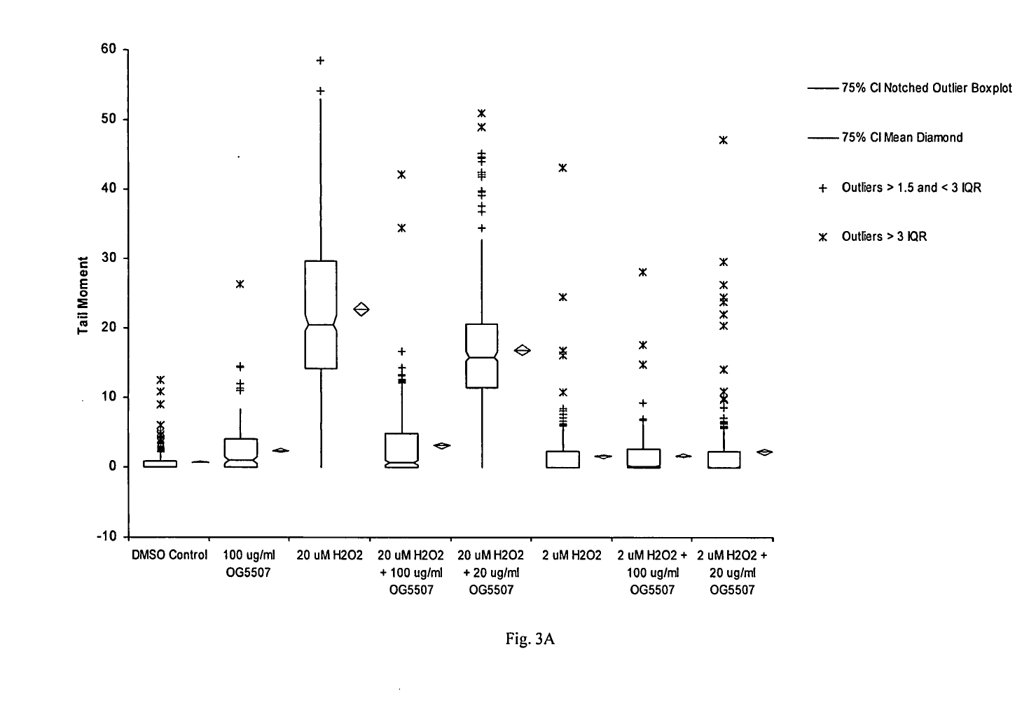

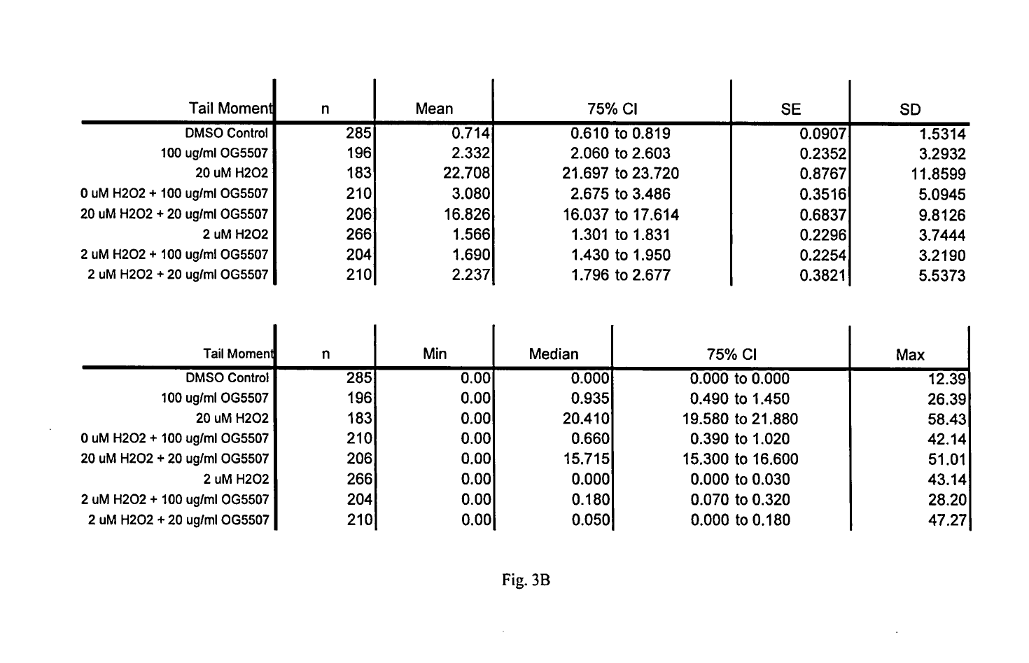

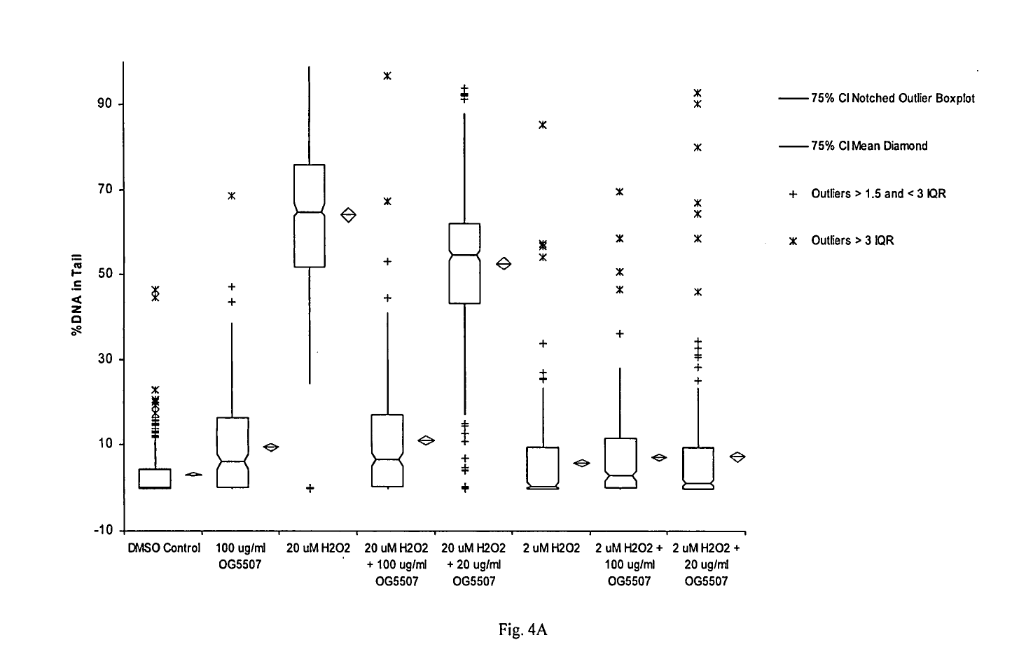

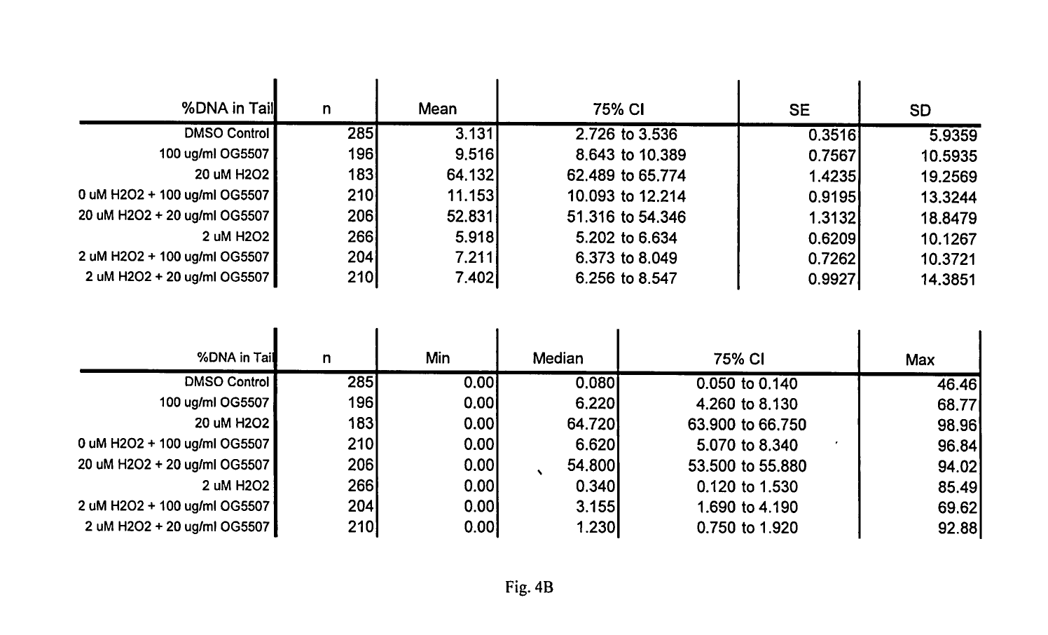

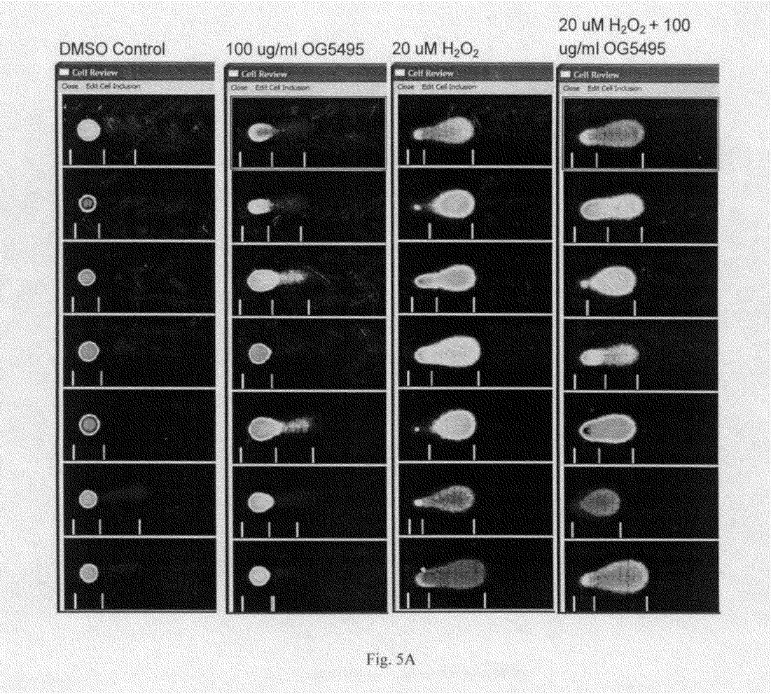

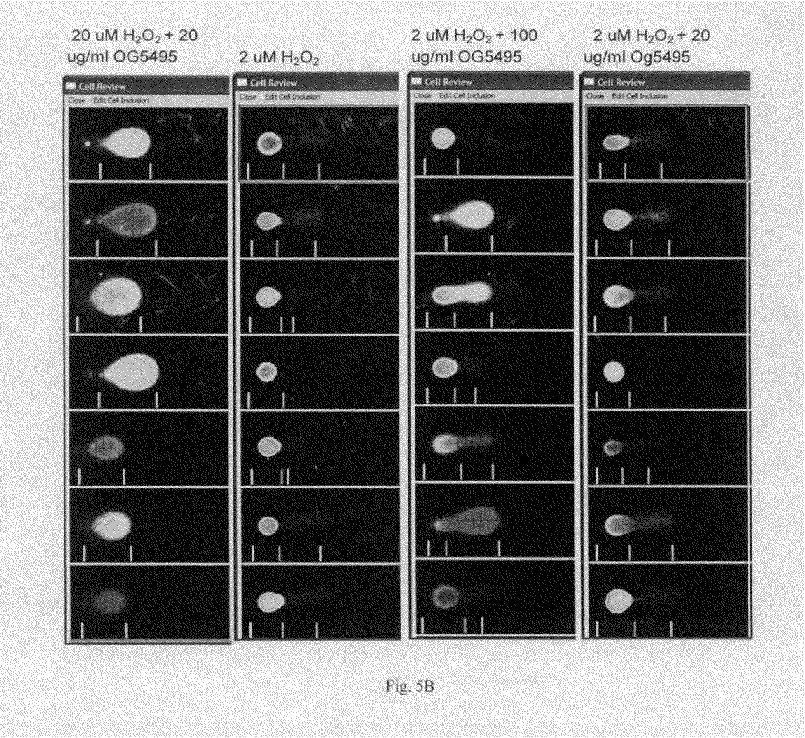

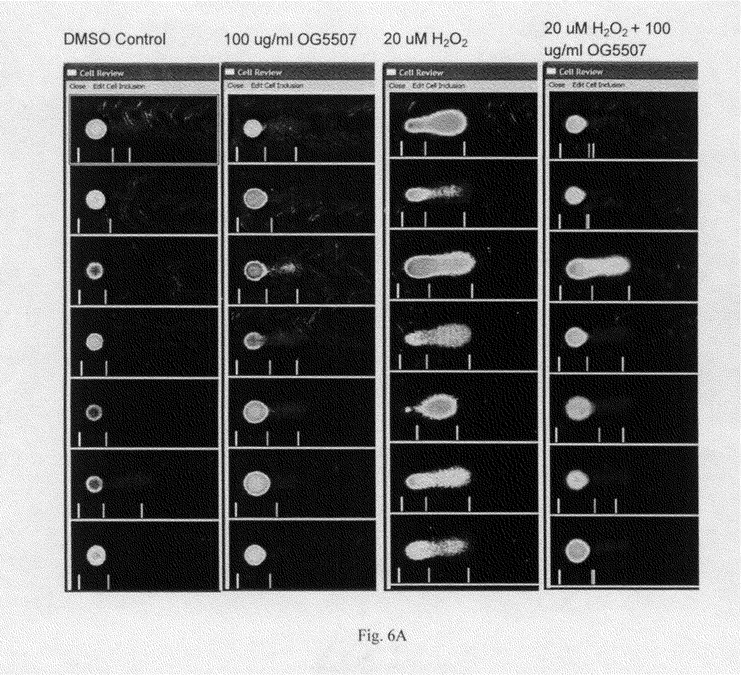

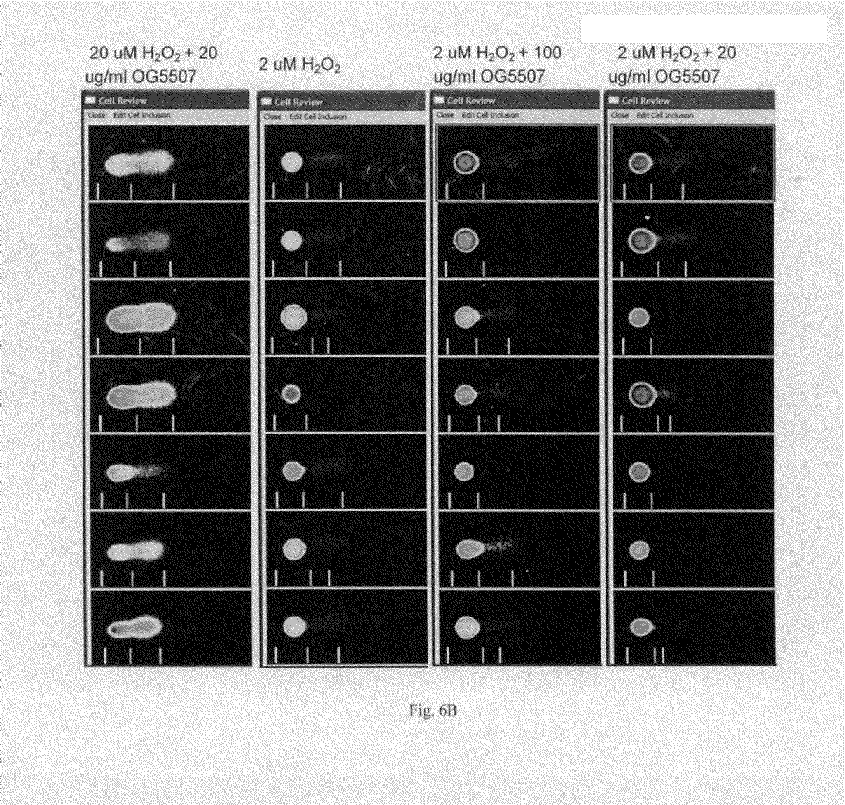

The Tail Moment and % DNA in the tail are presented graphically for OG5495 in FIGS. 1 and 2 [Panels A & B] respectively and for OG5507 in FIGS. 3 and 4 [Panels A & B] respectively. Comet assay images for OG5495 and OG5507 are presented in FIGS. 5 and 6 [Panels A & B] respectively.

The results from this example are also summarized in Tables 2 and 3 below.

TABLE 2
OG5495: Mean Tail Moment and % DNA in Tail + Std Error

| Compound | Sample | Date Run | Comets Scored | Tail Moment | % DNA in Tail |
|---|---|---|---|---|---|
| OG5495 | DMSO Control | Oct. 24, 2011 | 372 | 1.04 ± 0.13 | 3.68 ± 0.40 |
| OG5495 | 100 μg/ml compound | Oct. 24, 2011 | 269 | 10.10 ± 0.64 | 32.58 ± 1.55 |
| OG5495 | 20 μM H₂O₂ | Oct. 24, 2011 | 245 | 37.71 ± 0.69 | 88.52 ± 0.98 |
| OG5495 | 20 μM H2O2 + | Oct. 24, 2011 | 232 | 26.98 ± 0.83 | 72.15 ± 1.32 |
| | 100 μg/ml compound | | | | |
| OG5495 | 20 μM H2O2 + | Oct. 24, 2011 | 195 | 39.87 ± 0.77 | 92.27 ± 1.21 |
| | 20 μg/ml compound | | | | |
| OG5495 | 2 μM H₂O₂ | Oct. 24, 2011 | 194 | 3.25 ± 0.52 | 11.86 ± 1.27 |
| OG5495 | 2 μM H2O2 + | Oct. 24, 2011 | 149 | 15.29 ± 0.99 | 48.52 ± 2.15 |
| | 100 μg/ml compound | | | | |
| OG5495 | 2 μM H2O2 + | Oct. 24, 2011 | 213 | 5.27 ± 0.49 | 18.81 ± 1.45 |
| | 20 μg/ml compound | | | | |

TABLE 3
OG5507: Mean Tail Moment and % DNA in Tail + Std Error

| Compound | Sample | Date Run | Comets Scored | Tail Moment | % DNA in Tail |
|---|---|---|---|---|---|
| OG5507 | DMSO Control | Nov. 1, 2011 | 285 | 0.71 ± 0.09 | 3.13 ± 0.35 |
| OG5507 | 100 μg/ml compound | Nov. 1, 2011 | 196 | 2.33 ± 0.24 | 9.52 ± 0.76 |
| OG5507 | 20 μM H₂O₂ | Nov. 1, 2011 | 183 | 22.71 ± 0.88 | 64.13 ± 1.42 |
| OG5507 | 20 μM H2O2 + | Nov. 1, 2011 | 210 | 3.08 ± 0.35 | 11.15 ± 0.92 |
| | 100 μg/ml compound | | | | |
| OG5507 | 20 μM H2O2 + | Nov. 1, 2011 | 206 | 16.83 ± 0.68 | 52.83 ± 1.31 |
| | 20 μg/ml compound | | | | |
| OG5507 | 2 μM H₂O₂ | Nov. 1, 2011 | 266 | 1.57 ± 0.23 | 5.92 ± 0.62 |
| OG5507 | 2 μM H2O2 + | Nov. 1, 2011 | 204 | 1.69 ± 0.23 | 7.21 ± 0.73 |
| | 100 μg/ml compound | | | | |
| OG5507 | 2 μM H2O2 + | Nov. 1, 2011 | 210 | 2.24 ± 0.38 | 7.40 ± 0.99 |
| | 20 μg/ml compound | | | | |

### Example 2

### Effect of Phytonutrient Composition on OxLDL, MPO and PAI-1 Levels

Human Clinical Subjects and Protocol:

The purpose of this example was to demonstrate the simultaneous effects of the phytonutrient composition of the invention on OxLDL, MPO and PAI-1 Levels.

Fifteen (15) healthy volunteer subjects between 18-65 ages were screened and included in the study.

The clinical protocol used was IRB approved and the subjects were consented prior to both screening and the study visits (V_{N} where N is the visit number (i.e., first visit, second visit, etc.)).

Food Restrictions: Subject participant intake of food antioxidants was limited to only two servings total of fruits plus vegetables each day and not more than the amounts specified in Table 4 below.

TABLE 4
Food Antioxidant Food Restrictions

| | |
|---|---|
| Food not allowed | Coffee, chocolate, cocoa powder, tea, red wine, |
| | fruit juices |
| Food restricted | Beer—2 per day or white wine—1 glass per day |
| Fruits and | Apple/banana/pear (13), citrus fruit (1), beet/fennel |
| vegetables | cooked (1), zucchini/aubergines (eggplant) (2), |
| allowed | asparagus/string beans (2), mixed salad (7), tomatoes |
| | (2), tomato sauce (2), pesto sauce (1) |

The numbers in Table 4 describe the frequencies of consumption of allowed foods during study participation (i.e., maximum number of times food could be consumed as part of 14 serving limit for each week). Serving sizes were limited to 2 ounces of salad, 8 ounces of other kinds of vegetables on list, 8 ounces of apple, 8 ounces of pear, 5 ounces of banana, and 9 ounces of citrus fruit.

Food restrictions were for 2 weeks before the first blood draw. Following an overnight fast, blood specimens were collected in BD vacutainer tubes. The serum was separated and stored at −80° C. After the first blood draw, subjects consumed a high fat meal consisting of a bacon, egg and cheese sandwich, hash browns and 4 oz of whole milk/4 oz of Half and Half Another blood sample was collected one hour after ingestion of the high fat meal and the serum samples were stored at −80° C. Phytonutrient supplementation and diet restrictions were continued for 4 weeks and the blood was drawn in the BD vacutainer tubes before and after high fat meal and the serum was collected and stored at −80° C.

### Measurement of Plasminogen Activator Inhibitor-1 (PAI-1) in Serum Samples

Serum PAI-1 levels were measured using a solid phase ELISA kit (#KHC3071) from Invitrogen (Camarillo, Calif.). The PAI-1 levels were measured according to the protocols provided by manufacturer. The monoclonal antibody specific for human PAI-1 had been coated onto the wells. The serum was diluted with sample buffer (2 fold) and triplicates were used for each sample in a 96 well microtiter plate. The plates were read at 450 nM using a plate reader and the PAI-1 levels were measured using the standard supplied along with the kit. The data represent mean±SEM of 15 subjects. The significance difference was measured based on p values calculated using paired t test. *p<0.05 compared with V2 vs. V3.

### Measurement of Myeloperoxidase (MPO) in Serum Samples

Myeloperoxidase was measured using a solid phase two-site ELISA kit (#10-1176-01) from Mercodia (Uppsala, Sweden). MPO levels were measured according to the protocols provided by manufacturer. This immune assay is based on the sandwich technique in which two monoclonal antibodies are directed against separate antigenic determinants on the MPO-molecule. The serum was diluted for 5 times with sample buffer and triplicates were used for each sample in 96 well microtiter plate. The plates were read at 450 nM using a plate reader and the MPO levels were measured using the standards supplied along with the kit. The data represent mean±SEM of 15 subjects. The significance difference was measured based on p values calculated using paired t test. *p<0.05 compared with V2 vs. V3.

Measurement of Oxidized LDL (oxLDL) in Serum Samples

Oxidized LDL levels were measured using a solid phase two-site ELISA kit (#10-1143-01) from Mercodia (Uppsala, Sweden). OxLDL levels were measured according to the protocols provided by manufacturer. This immuno assay is based on the sandwich technique in which two monoclonal antibodies are directed against separate antigenic determinants on the oxidized apolipoprotein B molecule. The serum was diluted for with sample buffer (1:6561) and triplicates were used for each sample in 96 well microtiter plate. The plates were read at 450 nM using plate reader and the MPO levels were measured using the standard supplied along with the kit. The data represent mean±SEM of 15 subjects. The significance difference was measured based on p values calculated using paired t test. *p<0.05 compared with V2 vs V3.

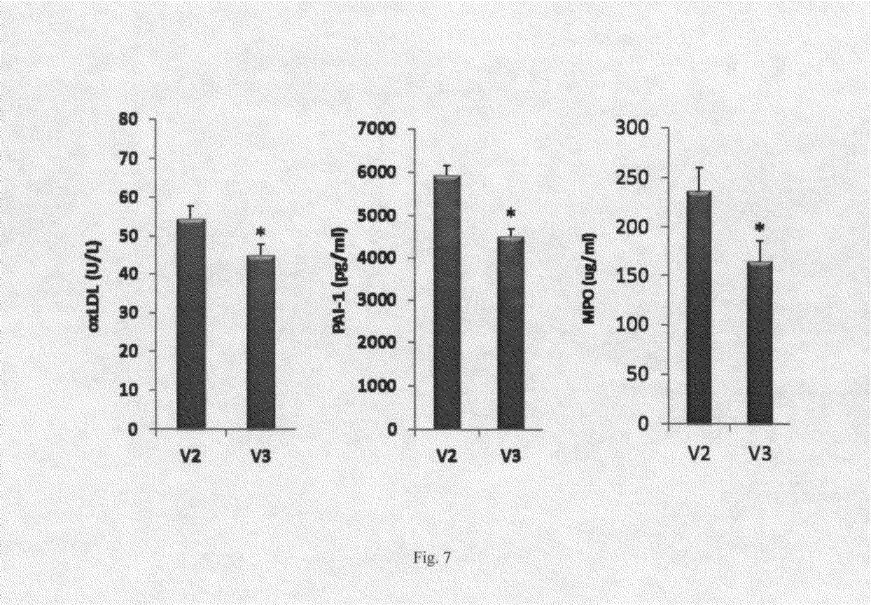

Results and Conclusions: Supplementation with the Phytonutrient composition of the invention for 4 weeks in humans concomitantly and significantly reduced serum oxLDL, PAI-1 and MPO levels (FIG. 7). Similarly, 4 week supplementation of phytonutrient concomitantly and significantly reduced serum oxLDL, PAI-1 and MPO levels compared to V2 vs. V3 visit (data not shown). This data suggests that the phytonutrient formulation could be beneficial for the treatment of atherosclerosis, stroke, cardiovascular diseases, diabetes, obesity, arthritis and asthma.

While the claimed invention has been described in detail and with reference to specific embodiments thereof, it will be apparent to one of ordinary skill in the art that various changes and modifications can be made to the claimed invention without departing from the spirit and scope thereof. Thus, for example, those skilled in the art will recognize, or be able to ascertain, using no more than routine experimentation, numerous equivalents to the specific substances and procedures described herein. Such equivalents are considered to be within the scope of this invention, and are covered by the following claims.

1. Use of a composition for simultaneously reducing serum OxLDL, MPO, and PAI-1 levels in a subject in need thereof, wherein said use comprises administering a composition comprising therapeutically effective amounts of two or more compounds or extracts selected from the group consisting of Acacia nilotica extract, artichoke extract, blueberry extract, catechins, chlorogenic acid, cinnamon, ellagic acid, grape seed extract, hesperidin, Momodica extract, prune extract, rosemary extract, and watercress extract and said composition is in a dosage form suitable for oral administration wherein said dosage form provides:
a. from 0.5 mg to 10 mg of Acacia nilotica extract;
b. from 0.5 mg to 10 mg of artichoke extract;
c. from 1 mg to 25 mg of blueberry extract;
d. from 2.5 mg to 25 mg of catechins;
e. from 25 mg to 75 mg of chlorogenic acid;
f. from 0.5 mg to 10 mg of cinnamon;
g. from 10 mg to 100 mg of ellagic acid;
h. from 2.5 mg to 50 mg of grape seed extract;
i. from 15 mg to 120 mg of hesperidin;
j. from 0.5 mg to 10 mg of Momodica extract;
k. from 0.5 mg to 10 mg of prune extract;
l. from 0.5 mg to 10 mg of rosemary extract; and
m. from 0.5 mg to 10 mg of watercress extract.

2. The use according to claim 1, wherein the composition provides three or more compounds or extracts selected from the group consisting of Acacia nilotica extract, artichoke extract, blueberry extract, catechins, chlorogenic acid, cinnamon, ellagic acid, grape seed extract, hesperidin, Momodica extract, prune extract, rosemary extract, and watercress extract.

3. The use according to claim 1, wherein the composition provides four or more compounds or extracts selected from the group consisting of Acacia nilotica extract, artichoke extract, blueberry extract, catechins, chlorogenic acid, cinnamon, ellagic acid, grape seed extract, hesperidin, Momodica extract, prune extract, rosemary extract, and watercress extract.

4. The use according to claim 1, wherein the composition provides five or more compounds or extracts selected from the group consisting of Acacia nilotica extract, artichoke extract, blueberry extract, catechins, chlorogenic acid, cinnamon, ellagic acid, grape seed extract, hesperidin, Momodica extract, prune extract, rosemary extract, and watercress extract.

5. The use according to claim 1, wherein the composition provides six or more compounds or extracts selected from the group consisting of Acacia nilotica extract, artichoke extract, blueberry extract, catechins, chlorogenic acid, cinnamon, ellagic acid, grape seed extract, hesperidin, Momodica extract, prune extract, rosemary extract, and watercress extract.

6. The use according to claim 1, wherein the composition provides seven or more compounds or extracts selected from the group consisting of Acacia nilotica extract, artichoke extract, blueberry extract, catechins, chlorogenic acid, cinnamon, ellagic acid, grape seed extract, hesperidin, Momodica extract, prune extract, rosemary extract, and watercress extract.
